(19) United States
(12) Patent Application Publication
(10) Pub. No.: US 2011/0045582 A1
(43) Pub. Date: Feb. 24, 2011

(54) METHODS AND APPARATUS FOR INTEGRATED CELL HANDLING AND MEASUREMENTS

(75) Inventors: Luke P. Lee, Livermore, CA (US); Jeonggi Seo, Albany, CA (US); Cristian Ionescu-Zanetti, Berkeley, CA (US); Michelle Khine, Berkeley, CA (US); Poorya Sabounchi, Pleasant Hill, CA (US); Robin Shaw, San Francisco, CA (US)

Correspondence Address: QUINE INTELLECTUAL PROPERTY LAW GROUP, P.C., P O BOX 458, ALAMEDA, CA, 94501, US

(73) Assignee: THE REGENTS OF THE UNIVERSITY OF CALIFORNIA, Oakland, CA (US)

(21) Appl. No.: 10/598,830

(22) PCT Filed: Mar. 14, 2005
(86) PCT No.: PCT/US05/08349

§ 371 Date: Jul. 16, 2010

Related U.S. Application Data
(60) Provisional application No. 60/552,892, filed on Mar. 12, 2004.

(51) Int. Cl. C12M 1/42; G01R 19/00; C23F 1/00

(52) U.S. Cl. 435/307.1; 324/76.11; 216/2

(57) ABSTRACT

Method and systems that provide improved cell handling and assays in microfluidic systems and devices particularly using lateral cell trapping and methods of fabrication of the same.

### CROSS REFERENCE TO RELATED APPLICATIONS

This application claims priority from PCT patent application PCT/US/05/008349 filed 14 Mar. 2005, which claims priority from provisional patent application 60/552,892, filed 12 Mar. 2004. These applications are incorporated herein by reference.

### COPYRIGHT NOTICE

Pursuant to 37 C.F.R. 1.71(e), applicants note that a portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to methods and/or system and/or apparatus involving analysis and/or handling of cells and/or other biological material and that can be adapted to other applications. In specific embodiments, the invention involves methods and/or system and/or apparatus involving various structures for manipulating objects, such as cells, in a fluidic medium and optionally performing certain manipulations thereof. In further specific embodiments, the invention involves methods, systems, and/or devices for patch-clamp analysis of cells and/or cell electroporation, and/or other cell assays or manipulation.

### BACKGROUND OF THE INVENTION

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

Handling, characterization, and visualization of individual cells has become increasingly valued in the fields of drug discovery, disease diagnoses and analysis, and a variety of other therapeutic and experimental work.

For example, the patch-clamp technique was described some 20 years ago to facilitate measurements and/or analysis of chemical and/or electrical properties at small regions of a cell membrane. Since then, patch clamp recording has had a profound impact on electrophysiology, playing a crucial role in the characterization of cellular ion channels. Traditionally, patch clamp recording is accomplished with a micromanipulator-positioned glass pipette under a microscope. As illustrated in FIG. 1A, a cell membrane patch is sucked into the glass pipette to form a high electrical resistance seal. Current that passes through the ion channels in either the membrane patch or the whole cell membrane is then recorded at different bias voltages. Despite improvements in the traditional patch clamp technique, it remains laborious.

Chip-based patch clamp devices have been proposed using silicon oxide coated nitride membranes, silicon elastomers, polyimide films, quartz or glass substrates. Recently, three dimensional structures more similar to patch pipettes have also been fabricated. Chip-based devices developed to date generally use a horizontal geometry as shown in FIG. 1B, where the patch pore is etched in a horizontal membrane dividing the top cell compartment from the recording electrode compartment.

Some more recent published patent applications that discuss various strategies related to patch-clamp analysis and related activities include the following U.S. applications, which are incorporated herein by reference to provide background.

| | | |
|---|---|---|
| 1 | 20040005696 | Substrate and method for measuring the electro-physiological properties |
| | | of cell membranes |
| 2 | 20030143720 | High throughput functional genomics |
| 3 | 20030139336 | Interface patch clamping |
| 4 | 20030138767 | Liquid interface configurations for automated patch clamp recording |
| 5 | 20030129581 | Patch-clamping method and apparatus |
| 6 | 20030065452 | High throughput functional genomics |
| 7 | 20030022268 | Method and apparatus for patch-clamp measurements on cells |
| 8 | 20020195337 | Polymeric electrode for electrophysiological testing |
| 9 | 20020182642 | Biosensors and methods of using the same |
| 10 | 20020064841 | Planar patch clamp electrodes |
| 11 | 20020045566 | Selective maxi-K potassium channel openers functional under conditions |
| | | of high intracellular calcium concentration, methods and uses thereof |

### OTHER REFERENCES

1. Asmild, M., Oswald, N., Krzywkowski, K. M., Friis, S., Jacobsen, R. B., Reuter, D., Taboryski, R., Kutchinsky, J., Vestergaard, R. K., Schroder, R. L., Sorensen, 11 C. B., Bech, M., Korsgaard, M. P. G. & Willumsen, N. J. (2003) Receptors & Channels 9, 49-58.
2. Bennett, P. B. & Guthrie, H. R. E. (2003) Journal of Biomolecular Screening 8, 660-667.
3. Dove, A. (2003) Nature Biotechnology 21, 859-864.
4. Entzeroth, M. (2003) Current Opinion in Pharmacology 3, 522-529.
5. Fertig, A. Tilke, R. H. Blick, J. P. Kotthaus, J. C. Behrends, and G. ten Bruggencate, Applied Physics Letters 77, 1218-1220 (2000).
6. Fertig, M. Klau, M. George, R. H. Blick, and J. C. Behrends, Applied Physics Letters 81, 4865-4867 (2002).
7. Fertig, N., Blick, R. H. & Behrends, J. C. (2002) Biophysical Journal 82, 3056-3062.
8. Immke, D. & Korn, S. J. (2000) Journal of General Physiology 115, 509-518.
9. Klemic, K. G., Klemic, J. F., Reed, M. A. & Sigworth, F. J. (2002) Biosensors and Bioelectronics 17, 597-604.
10. Lehnert, T., Gijs, M. A. M., Netzer, R. & Bischoff, U. (2002) Applied Physics Letters 81, 5063-5065.
11. Lin, T. Kerle, and T. P. Russel, Macromolecules 35, 3971-3976 (2002).
12. Neubert, H. J. (2004) in Analytical Chemistry, Vol. 76, pp. 327a-330a.
13. Sakmann, B. & Neher, E. (1984) Annual Review of Physiology 46, 455-472.
14. Stett, A., Burkhardt, C., Weber, U., van Stiphout, P. & Knott, T. (2003) Receptors & Channels 9, 59-66.
15. Stett, V. Bucher, C. Burkhardt, U. Weber, and W. Nisch, Medical & Biological Engineering & Computing 41, 233-240 (2003).
16. Thorsen, T., Maerkl, S. J. & Quake, S. R. (2002) Science 298, 580-5
17. Trapani, J. G. & Korn, S. J. (2003) Bmc Neuroscience 4, -.
18. Wang, X. B. & Li, M. (2003) Assay and Drug Development Technologies 1, 709-717.
19. Wood, C., Williams, C. & Waldron, G. J. (2004) in Drug Discovery Today, Vol. 9, pp. 434-441.
20. Xu, J., Wang, X. B., Ensgn, B., Li, M., Guia, A. & Xu, J. Q. (2001) Drug Discovery Today 6, 1278-1287.

### SUMMARY

The present invention, in specific embodiments, involves methods and or devices that provide improved cellular handling using lateral cell trapping junctions at a micron scale integrated with microfluidic channels. Cell immobilization or trapping pores in specific embodiments generally are arranged as openings in a sidewall or analogous structure of a main fluidic channel. At times herein, this geometry is referred to as a lateral pore or junction.

The present invention, in further embodiments, involves an integrated multiple cellular handling array system or device that utilize lateral cell trapping junctions. In specific example systems, the intersectional design of a microfluidic network provides multiple cell addressing and manipulation sites for efficient electrophysiological measurements and cell manipulations at a number of sites.

The device geometry according to specific embodiments of the invention not only minimizes capacitive coupling between the cell reservoir and the patch channel, but also allows for visual observation of membrane deformation. In specific embodiments, device fabrication is based on micromolding of one or more elastomers (such as, polydimethylsiloxane (PDMS)), allowing for inexpensive mass production of disposable high-throughput biochips. Other embodiments can be constructed from bonded silicon/polysilicon surfaces or injection molded polymers.

In specific embodiments, the geometry not only minimizes capacitive coupling between the cell reservoir and the channel, but also allows for simultaneous optical and electrical characterization. Further, in specific embodiments, the device geometry, together with the low dielectric constant of a preferred elastomer, e.g., PDMS, results in very low capacitive coupling between the cell reservoir and the channel.

The lateral design according to some specific embodiments of the invention also allows for construction of systems having efficient multiplexing of measurements, exchange of intracellular and extracellular electrolyte while the cell is attached to the pore, and optical observation of membrane deformation and cellular content. Thus, in specific embodiments, the invention enables high throughput, low cost cell-based patch clamp measurements and other cellular manipulations and/or assays.

According to specific embodiments of the invention, aspects of the invention can be incorporated into one or more integrated systems that provide simple yet elegant means for trapping multiple cells instantaneously by pneumatic controls and allows simultaneous electrical and optical characterizations, providing an ideal mechanism for high throughput screening (HTS) single cells analysis and drug discovery.

In further specific embodiments, the novel methods and devices according to specific embodiments of the invention can be used in various micrometer systems. Applications include BioMEMS, lab on a chip, cell-based assays, etc.

While example systems according to specific embodiments of the present invention are described herein as used for performing testing or characterizations of biological cells, it will be understood to those of skill in the art that a detector according to specific embodiments of the present invention can be used in a variety of applications for manipulating and assaying devices at a roughly cellular size (100 nm-40 μm). These applications include, but are not limited to: chemical systems; testing for contaminants in foodstuffs; detecting the presence of a desired substance or desired reaction, etc.

### OTHER FEATURES & BENEFITS

The invention and various specific aspects and embodiments will be better understood with reference to drawings and detailed descriptions provided in this submission. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

In some of the drawings and detailed descriptions below, the present invention is described in terms of the important independent embodiment of a biologic assay and/or array system and components thereof. This should not be taken to limit the invention, which, using the teachings provided herein, can be applied to a number of other situations.

In some of the drawings and detailed descriptions below, the present invention is described in terms of a number of specific example embodiments including specific parameters related to dimensions of structures, pressures or volumes of liquids, or electrical values. Expect where so provided in the attached claims, these parameters are provided as examples and do not limit the invention to other devices or systems with different dimensions.

All references, publications, patents, and patent applications cited in this submission are hereby incorporated by reference in their entirety for all purposes.

### BRIEF DESCRIPTION OF THE DRAWINGS

FIG. 1 illustrates a comparison of some prior patch clamp setups including (a) traditional patch clamp using a glass micropipette and (b) an on-chip horizontal planar patch clamp.

FIG. 2A-C illustrate example aspects of cell manipulation devices according to various embodiments of the invention.

FIG. 3 illustrates aspects of an example double channel clamp allowing for rapid change of intracellular and extracellular solutions according to alternative specific embodiments of the invention.

FIG. 4 illustrates an example of a simple lateral junction cell trapping disposable concentric clamp according to alternative specific embodiments of the invention.

FIG. 5A-B illustrate optional different geometries for an opening to a lateral cell junction according to specific embodiments of the invention.

FIG. 6 illustrates aspects of an example integrated cellular manipulation array on a microfluidic platform according to specific embodiments of the invention.

FIGS. 7A and B illustrate an example configuration of an integrated cell handling system having lateral trapping junctions and multiple microfluidic control layers according to specific embodiments of the invention.

FIG. 8 is a block diagram illustrating an example of operation of an integrated cell handling device according to specific embodiments of the invention.

FIG. 9A-D show four frames from a micrograph movie showing a HeLa cell being trapped at a lateral junction by applying a negative pressure (e.g., 2 psi) to a trapping channel according to specific embodiments of the invention.

FIG. 10 illustrates an example of fabrication of a device array according to specific embodiments of the invention.

FIGS. 11A&B illustrate current response to a 20 mV voltage pulse before (a) and after (b) cell trapping of an example device according to specific embodiments of the invention.

FIG. 12 illustrates various images of aspects of a cell trapped at the orifice of a capillary according to specific embodiments of the invention.

FIG. 13 is a diagram showing an example of whole mammalian cell currents recorded according to specific embodiments of the invention.

FIG. 14 is a schematic representation of a basic design unit for fast reagent application and removal according to specific embodiments of the invention.

FIG. 15 is a schematic representation showing an example methodology for the application of multiple reagents to a target sample area according to specific embodiments of the invention. (A state where channel n4 is on is shown).

FIG. 16 is a schematic representation showing an example methodology for applying an arbitrary concentration to a sample area according to specific embodiments of the invention. (A microfluidic mixer is being driven by input pressures/flow rates in this example.)

FIG. 17 is a schematic representation showing use of trapping channels for immobilizing cells in the target area for fast reagent application and removal according to specific embodiments of the invention.

FIG. 18 is a schematic representation of a basic design unit for interrogation of adherent cells attached to the sample area substrate allowing for fast reagent application and removal according to specific embodiments of the invention.

FIG. 19 is a schematic representation of a basic design for applying a variable pressure to the injection channel by employing two pressure reservoirs and a solenoid valve according to specific embodiments of the invention.

FIG. 20 illustrates an example schematic of a hollow electrode interface (in this example configured on a PCB) mating with device and tubing according to specific embodiments of the invention.

FIG. 21 illustrates a bottom view of an example hollow electrode interface according to specific embodiments of the invention.

FIG. 22 illustrates an exploded side view of tubing, interface and device assembly of an example hollow electrode interface according to specific embodiments of the invention.

FIG. 23 is a block diagram showing a representative example logic device in which various aspects of the present invention may be embodied.

FIG. 24 (Table 1) illustrates an example of diseases, conditions, or statuses that can evaluated or for which drugs or other therapies can be tested according to specific embodiments of the present invention.

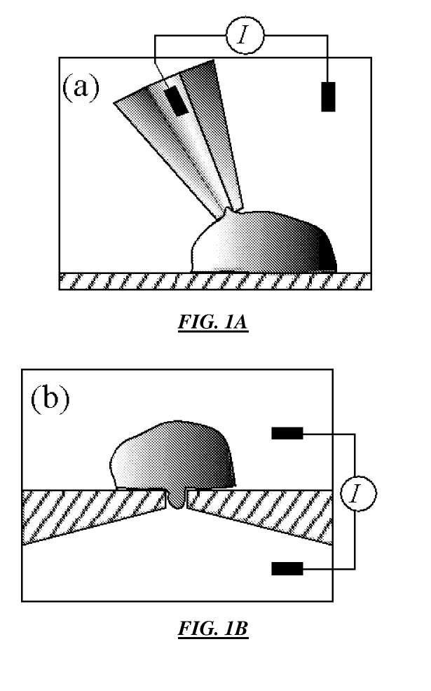

### DESCRIPTION OF SPECIFIC EMBODIMENTS

### 1. Overview

Traditional patch clamp recording is accomplished in a vibration free environment by using a micromanipulator to position the tip of a glass pipette against the membrane of a cell. Carefully applied negative pressure through the pipette tip causes the membrane to invaginate into the pipette and a generally gigaohm or greater resistance seal to form between the pipette and cell. For whole cell recording, a second application of negative pressure or electrical current ruptures the captured membrane, providing continuity between the pipette electrode and the cell's cytoplasm. In voltage clamp mode, the membrane is clamped to a preset potential, and the current required to maintain this potential is recorded. Current recordings with different electrical protocols and in the presence of different reagents are used to characterize ion channel properties.

Despite the success of the traditional patch clamp technique, it requires complex and expensive setups and remains highly laborious. Thus, the patch clamp technique is limiting in proteomics and drug development screens, which demand high-throughput automated measurements.

Chip-based patch clamp devices have been proposed using micromachined substrates from glass, quartz, coated silicon nitride, and treated elastomers. Some devices have been proposed for commercial platforms for pharmaceutical drug screening and drug safety testing. In general, these devices use a horizontal planar geometry, where the recording capillary is etched in a horizontal membrane dividing a top cell compartment from a bottom recording electrode compartment. Variations of this configuration include nozzle designs consisting of a raised region around the circumference of the patch orifice. This horizontal planar patch approach generally has a limitation on the density of cell trapping sites and limited ability to visualize and manipulate cells being studied. In some systems, there is a relatively slow delivery of reagents to the cells and in a number of systems a need to preserve a vibration free environment.

PDMS-based patch clamps have been reported for performing whole cell recording of Xenopus oocytes by micromolding of an orifice in a planar substrate. A limitation of this design is the large size of the recording orifice as positioned in the planar substrate, which may be suitable for whole cell recording of large oocytes but not for smaller mammalian cells. A second limitation of this technique is the requirement for oxygen plasma treatment prior to use in order to obtain adequate seals as the plasma treatment is unstable, and requires device use within hours of manufacture. Non-elastomer devices have been reported for performing whole cell recording of mammalian cells by employing a planar design with vertical lithographically defined pores in silicon and other substrates. Integration of planar patch designs with microfluidics remains a technologically challenging task.

Some proposals for automated patch clamp technology do not allow for easy visual access to the cells being studied. Even where the cells are visually accessible, devices in which the geometry is not sufficiently dense do not allow easy viewing of multiple cells using straightforward single microscope techniques.

The present invention, according to various specific embodiments as described herein, provides methods, systems, and devices that allow for very dense integration of cell handling and microfluidic control in an integrated platform and that allow for inexpensive manufacture, easy of visualization, and other advantageous as will be apparent from the descriptions herein.

### 2. Example Device Configurations

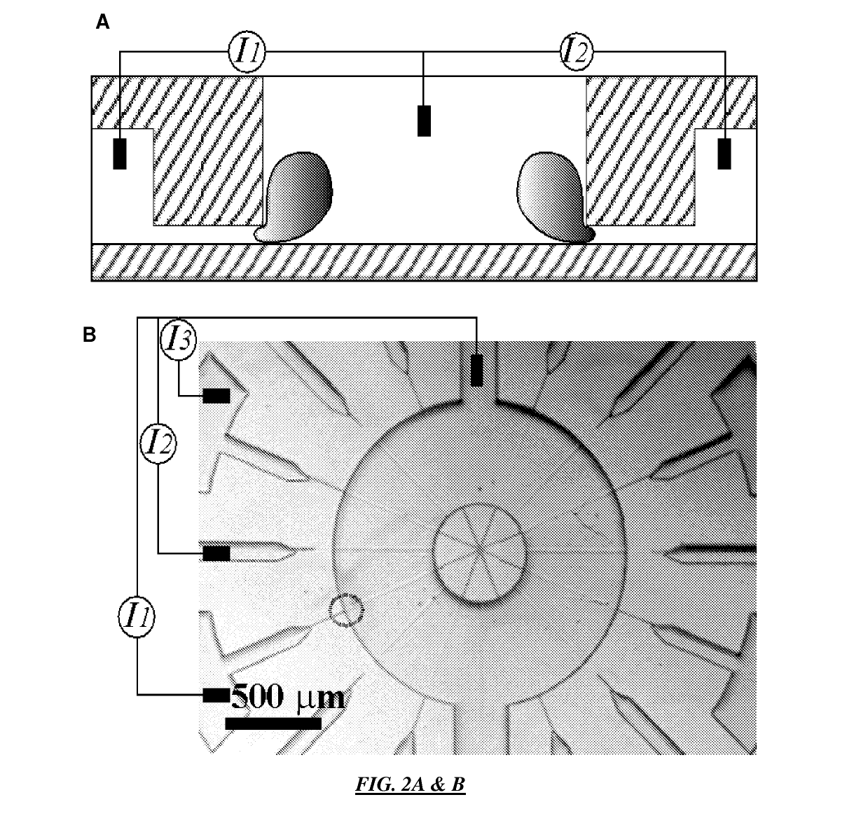

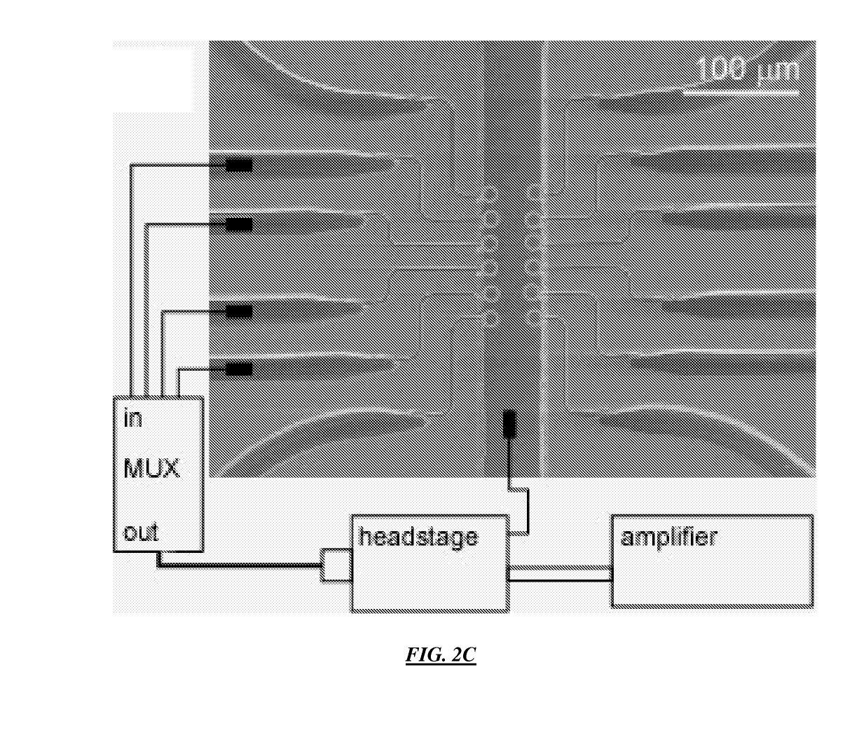

FIG. 2A-C illustrate example aspects of cell manipulation devices according to various embodiments of the invention. Each of the examples shows lateral cell trapping junctions according to specific embodiments of the invention. These junctions dramatically reduce capacitive coupling between the cell reservoir and the channel, an important feature for example for low noise patch channel recording. With the pores in the horizontal plane, multiplexed parallel sites that are only about 10-20 microns apart are possible. Channel binding drugs can therefore be administered in small volumes, while the effects on channel activity can be recorded and/or viewed in parallel at a number of sites. In various example fabrications, the whole device is fabricated using micromolding of an elastomer such as polydimethylsiloxane (PDMS), a high-throughput, inexpensive procedure.

More specifically, FIG. 2A is a schematic diagram showing in cross-section two lateral cell trapping pores or junctions on the sides of a central channel. In this and other figures herein, specific examples are shown wherein a narrow trapping fluidic connecting operatively connects to the lateral pore. A larger fluidic connection or reservoir is provided at the other end of the narrow trapping connection (or channel) to allow easier fluidic access. Such trapping connections and pores can be configured in a circular central channel, a curved central channel, or a roughly linear central channel, as described elsewhere herein. Spacing between pores and the dimensions of elements can be varied, as will be understood to those of skill in the art from the teachings described herein.

FIG. 2B is a top view micrograph of an example device showing a circular central channel with 14 radial cell trapping pores. Each trapping pore is connected by a narrow trapping channel to a fluidic reservoir that can be connected to other microfluidic controls on the device as described elsewhere herein. In this example, two larger channels that can be used for ingress or egress of cells, fluids, or other materials connect to the central channel. The operational electrical connectivity of an optional reference electrode in one of the ingress/egress channels and three patch electrodes is shown schematically in the figure. The small circle at the lower left indicates one of the lateral capture sites. In this design, cell pores are radially about 200 microns apart, as shown by the dimensional bar. This dimension is an example only, and much smaller versions of this same design can also be constructed.

FIG. 2C is a top view micrograph of an example higher density device showing a roughly linear main channel, and 12 trapping pores and connected trapping channels that are about 20 microns (μm) apart or less. Again, optional electrical connectivity to recording apparatus is schematically shown. In this figure, the 12 circular diagrams indicate locations of trapped cells at the lateral trapping junctions.

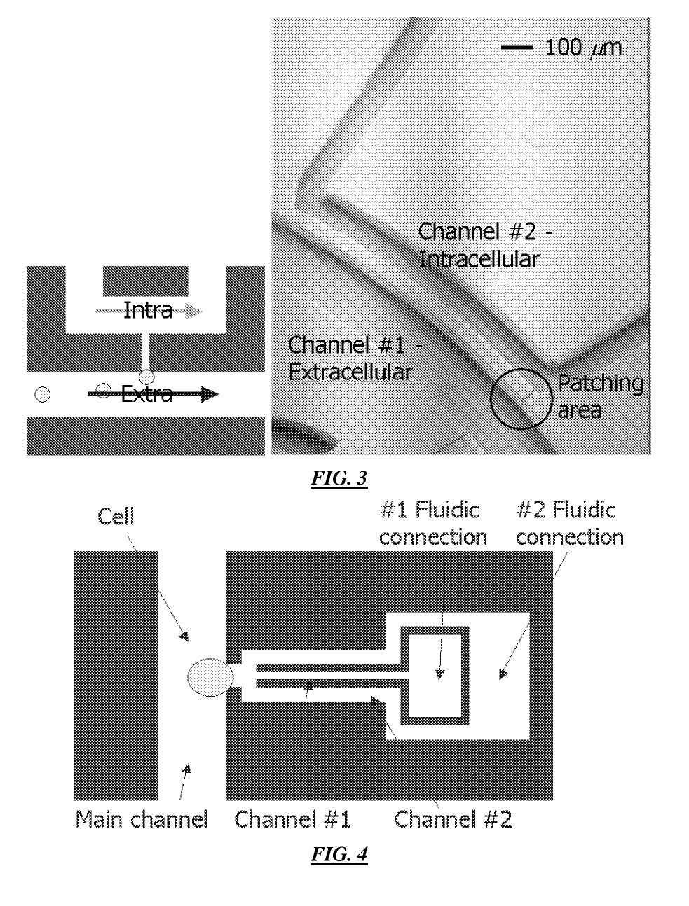

FIG. 3 illustrates aspects of an example double channel clamp allowing for rapid change of intracellular and extracellular solutions according to alternative specific embodiments of the invention. In this alternative design, negative pressure can be maintained in the intracellular channel with respect to the extracellular channel to cell trapping, while the two separated channels allow rapid exchange of fluids in either channel. This type of trapping junction can be incorporated in various designs as will be understood from the teachings provided herein.

FIG. 4 illustrates an example of a simple lateral junction cell trapping disposable concentric clamp according to alternative specific embodiments of the invention. This is a further alternative configuration showing two fluidic connections for the intracellular area, allowing for fast fluidic exchange.

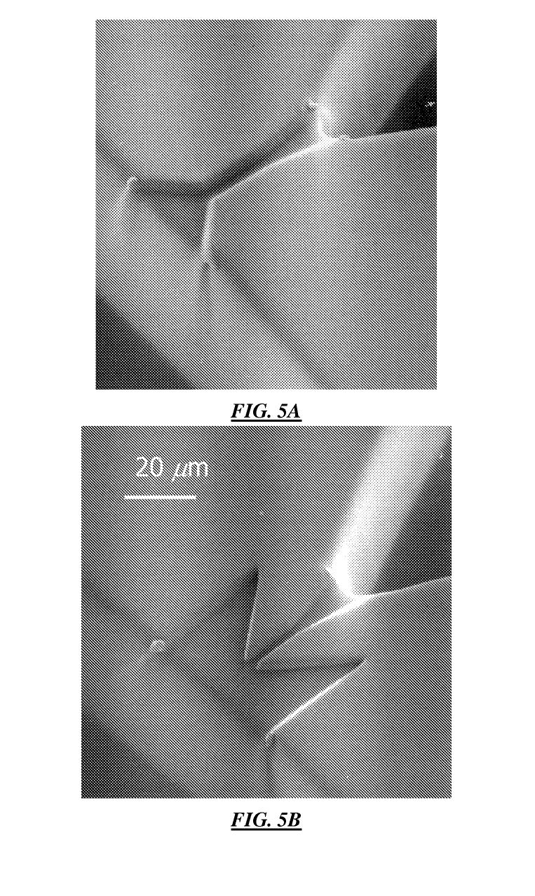

FIG. 5A-B illustrate optional different geometries for an opening to a lateral cell junction according to specific embodiments of the invention. In each of these figures, a different geometry opening is presented to the cellular area for effecting cell trapping. Either of these junctions or other junction configurations including semi-circular capture areas can be incorporated into devices according to specific embodiments of the invention as an alternative to the straight lateral capture pore illustrated in most of the examples provided herein. While different configurations may provide better trapping and/or sealing in particular situations, the pore opening directly into the cell channel is a presently preferred embodiment.

### 3. Example Integrated Cell Manipulation System

In further embodiments, a high-density integrated cell handling system according to specific embodiments of the invention provides improves visualization and control of cell position. In specific embodiments, the invention provides for the integration of whole cell electrophysiology with easily manufactured microfluidic lab-on-a-chip devices. As described elsewhere herein, in specific embodiments, such devices are fabricated by micromolding of polydimethylsiloxane (PDMS) or materials with similar or analogous properties. In specific embodiments, holding a cell at a pore at an integrated substrate eliminates the need for vibration isolation as compared to pipette-based recording. The present invention in specific embodiments achieves this even with very dense pore placement. According to specific embodiments, a cell manipulation device of the invention also allows direct cell visualization including of multiple cells and using standard microscopy.

In further example embodiments, microfluidic integration allows recording capillaries to be arrayed about less than 10-30 microns apart, for a total chamber volume of less than about 0.5 nanolitres. The geometry of recording capillaries in specific embodiments permits high quality, stable, whole-cell seals despite the hydrophobicity of some surfaces, such as PDMS. In particular embodiments, the lateral geometry of the trapping junctions in combinations with the long, thin trapping channels as shown provides great surface area allowing for a more stable seal.

Replacing silicon micromachining with PDMS micromolding has a number of advantages, among which, the fabrication is sufficiently simple (requiring only molding and bonding) and economical to enable the production of single use disposable devices. Further, unlike silicon based devices, the PDMS device is transparent and can be bonded to a 12 mm glass coverslip, permitting placement on the stage of an inverted microscope and visualization of cells during recording.

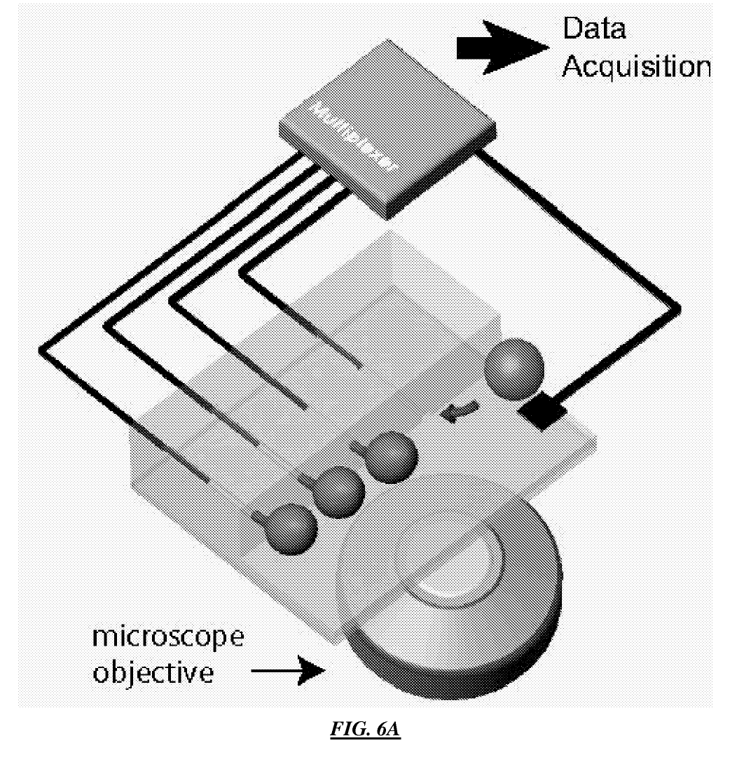

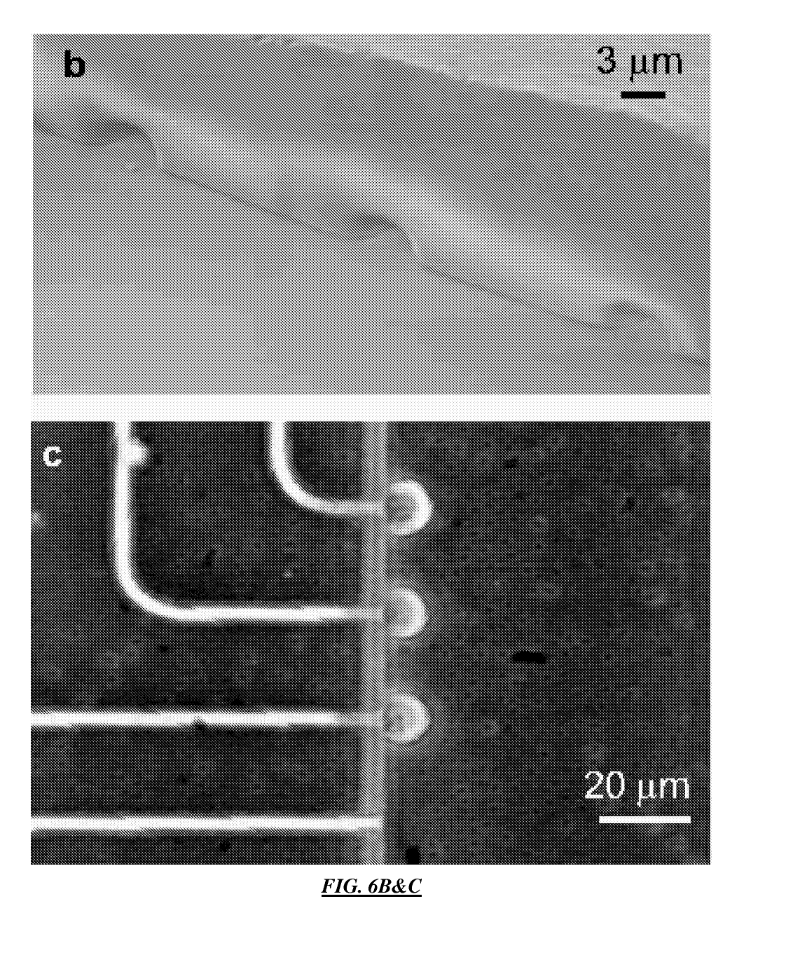

FIG. 6 illustrates aspects of an example integrated cellular manipulation array on a microfluidic platform according to specific embodiments of the invention. As above, cell trapping is achieved by applying negative pressure to recording capillaries which open into a main chamber containing cells in suspension, which are represented schematically as spheres in FIG. 6A. Attached cells deform, protruding into the capillaries.

Where desired, patch clamp recordings are obtained by placing electrodes (such as the AgCl electrodes described herein) in each of the capillaries, as well as the main chamber. Signals are fed through a multiplexing circuit and into the data acquisition system (multiplexer setup and microscope objective not to scale). The device can be bonded to a glass coverslip for optical monitoring.

FIG. 6B is a scanning electron micrograph of three recording capillary orifices as seen from a main chamber of a cell manipulation system according to specific embodiments of the invention. Example capillary cross-section dimensions are 4 μm×3 μm, with a pore-to-pore (or site-to-site) distance of about 20 μm.

FIG. 6C is a darkfield optical microscope image of cells trapped at three capillary orifices. An example device having 12 capillaries arrayed six along each side of the main chamber fluidic channel, along a 120 μm distance is shown in the micrograph of FIG. 2C.

Advantageous features of this design are inherent microfluidic integration, very high density of patch sites, and the ability to measure both cell deformation and membrane integrity during electrical recording. Accurate recording of ionic currents was validated through a head to head comparison with traditional electrophysiology. Pharmaceutical modification of ion channel availability was also demonstrated by measuring changes in ion channel currents after the introduction of the K₊ channel blocker TEA into the extracellular solution. The ability of soft lithography to produce a microfluidic platform for high throughput electrophysiology demonstrated by the invention makes possible the seamless integration of additional functionalities such as cell culture, cell sorting and the analysis of protein content in a single disposable device.

Experimental results have shown what is believed to be the first whole cell recording of mammalian cells on an inexpensive microfluidic platform. Mammalian cells provide numerous challenges in handling due in part to their small size as is understood in the art. On example of experimental results indicate that recordings of activation of the voltage sensitive potassium channel Kv2.1 in mammalian cells compare well with traditional pipette recordings.

As a further improvement to a microfluidics based lateral geometry for electrical measurements of cells, the average seal resistance is increased to better obtain a whole cell configuration and patch clamp recording, for example by partial cure bonding of the elastomer, which results in improvement in cell attached seal resistance to the gigaohm range. Thus, according to specific embodiments, the invention comprises a novel multi-channel (e.g., 12) patch clamp array, which is less than about 0.5 nL in total volume and incorporates partial cure bonding, yielding robust seals on mammalian cells. Seals are established without the need of vibration isolation equipment.

### Larger-Scale Integrated Example

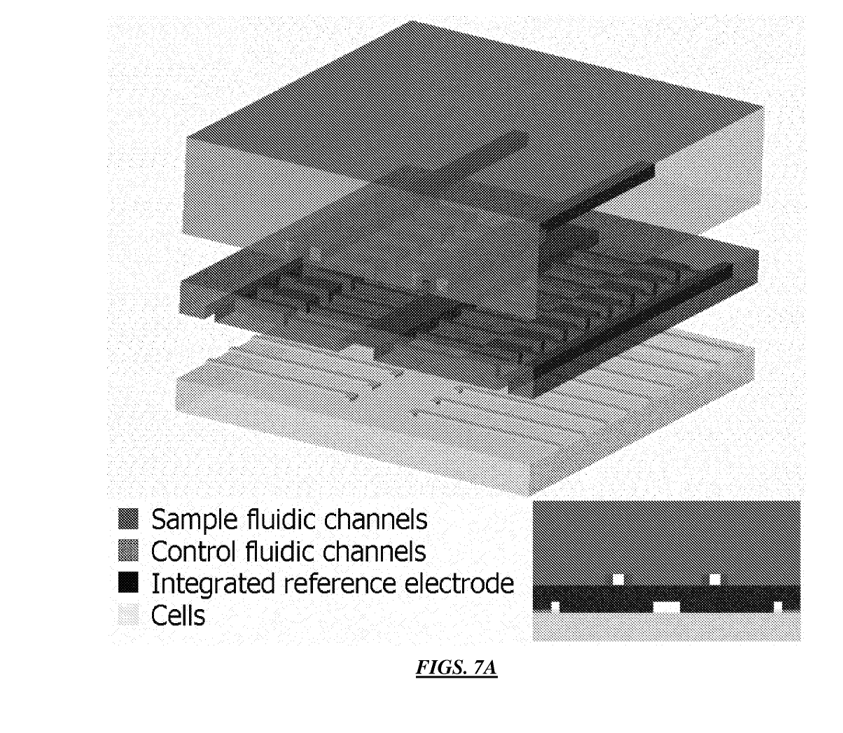

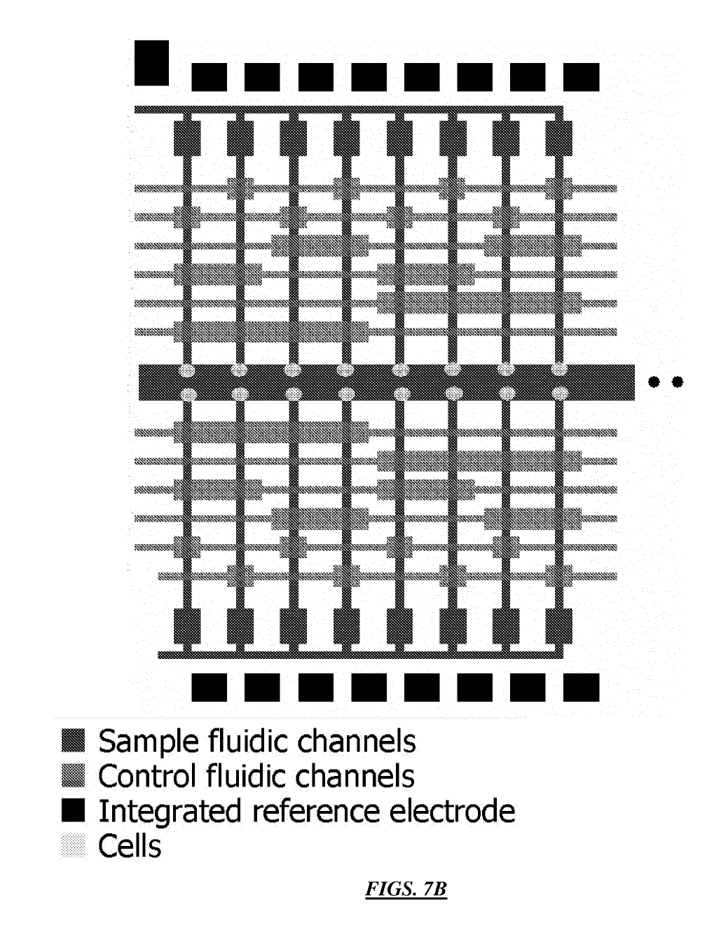

In further embodiments, lateral trapping junctions of the invention can be embodiment into larger-scale integrated systems for high-throughput cellular analysis. FIGS. 7A and B illustrate an example configuration of an integrated cell handling system having lateral trapping junctions and multiple microfluidic control layers according to specific embodiments of the invention. This example system illustrates a fully integrated system with linear central chambers. Patterned recording electrodes (shown in gold) are arranged on the lowest (green) substrate, which can be made of any suitable material, e.g., silicon, glass, ceramic, etc. Trapping channels and a central channel are integrated with elastomer valving sites in a middle layer. A valve channel control layer is configured on the top. In FIG. 7B, 14 cells are diagrammatically shown trapped in a linear channel at lateral junctions by operation of the control fluidic channels.

### 4. Example Operation

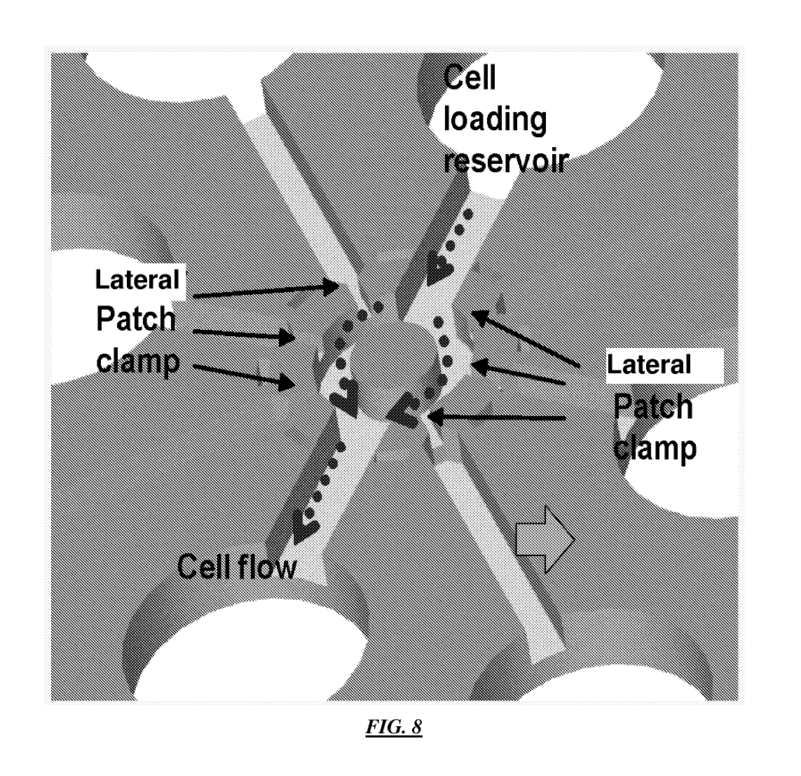

FIG. 8 is a block diagram illustrating an example of operation of an integrated cell handling device according to specific embodiments of the invention. This diagram shows one example of how cells can be loaded from a cell reservoir and trapped at pores using negative pressure at one or more cell trapping channels as described herein. This general operational design can be adapted to any configuration of cell manipulation devices according to the invention, including circular, linear, or curved. This general operational design allows easy cell trapping; easy optical characterizations; simple cell loading for multiple single cell analysis; high throughput, and small drug usage.

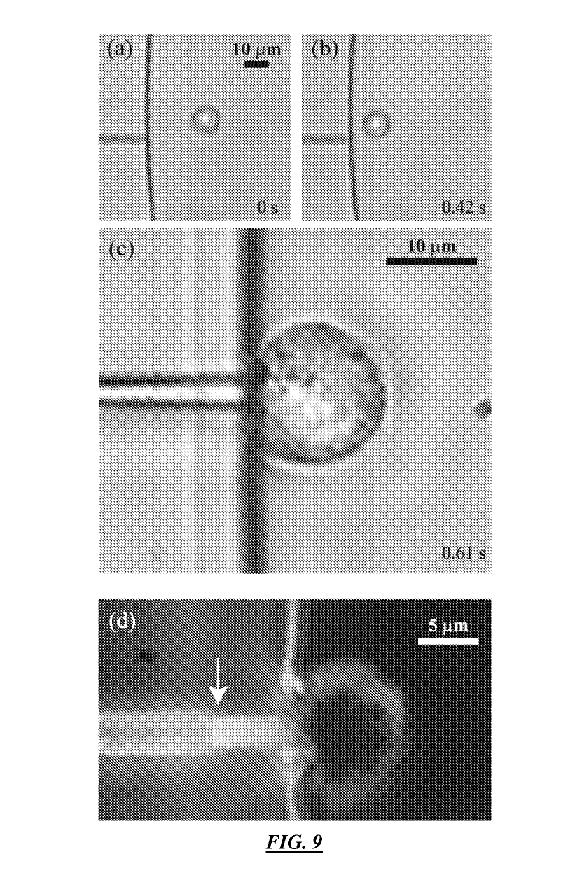

FIG. 9A-D show four frames from a micrograph movie showing a HeLa cell being trapped at a lateral junction by applying a negative pressure (e.g., 2 psi) to a trapping channel according to specific embodiments of the invention. In FIG. 9C, the frame is magnified in order to show cell positioning on the pore. In FIG. 9D, real time observation of the cell membrane deformation is shown. In this example figure, pore openings are approximately 1-3 microns in cross-section.

Further details of example operation of devices and/or systems according to specific embodiments of the invention are discussed in the experimental results section below.

### 5. Example Fabrication

Systems and devices as described herein can be fabricated using any techniques or methods familiar from the field of photolithography, nano-fabrication, or micro-fluidic fabrication. For completeness of this disclosure and to discuss additional and independent novel aspects according to specific embodiments of the invention, specifics of example fabrication methods are provided below.

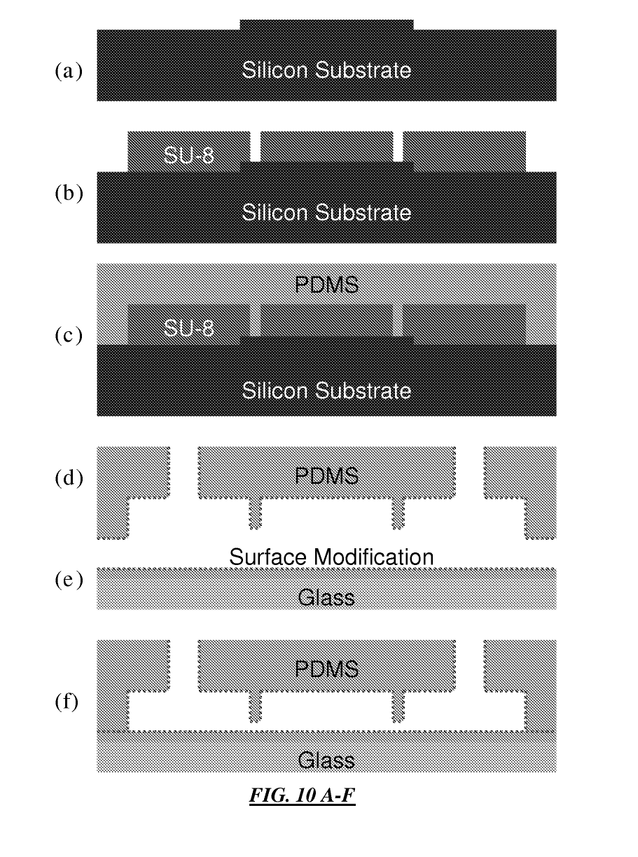

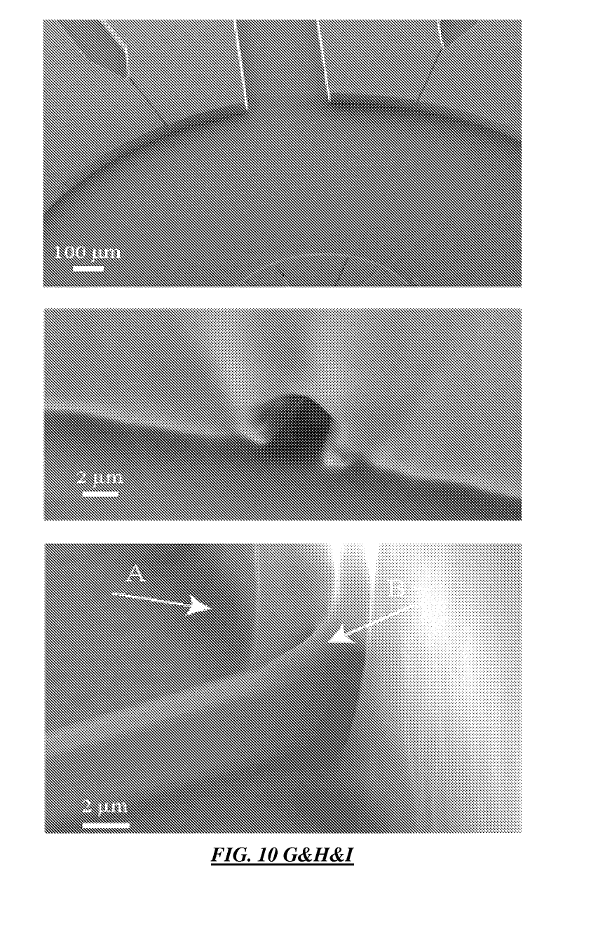

Example fabrication steps according to specific embodiments of the invention are shown diagrammatically in FIG. 10(a-f). In this example, a silicon or other suitable substrate mold is prepared using surface micromachining and/or photolithography techniques. In other examples, any other appropriate material and any other techniques for making a mold could be used.

In a micromachining example according to specific embodiments of the invention, first, 3.1 μm height patterns are made, defining the narrow cell trapping channels using deep reactive ion etching (FIG. 10A). Second, 50 μm high patterns are added for wide connection regions using SU-8 negative photoresist (FIG. 10B). After a base and a curing agent of PDMS were mixed (1:10), the liquid mixture is poured onto the mold and cured at 80° C. for 1 hour (FIG. 10C).

After the PDMS is cured, the devices are detached and can be mechanically punched. In this specific example, the devices and the glass substrate pre-coated with a thin PDMS layer are treated with oxygen plasma (FIGS. 10D and E) and the devices are bonded to the thin PDMS (FIG. 10F).

SEM images of overall device geometry before bonding (upside down) and a closeup of the patch pore after bonding are shown in FIGS. 10G and H. A SEM image of the mold is shown in FIG. 10I.

In this example, it was observed that the top of the orifice is rounded. The rounding of the top of the orifice is a beneficial result of mold fabrication, and it was observed that the channel top is rounded next to the patch orifice in the mold (FIG. 10I). When the SU8 is selectively polymerized in order to create the large channels on top of the small patch channel defined in Si, light scattering near the Si surface results in a deviation from the intended vertical SU8 wall. The resulting rounded feature at the bottom of the SU8 wall (FIG. 10I, arrow A) is also present on top of the small Si wall (FIG. 10I, arrow B), resulting in rounding of the patch orifice top. This feature is reproducible in specific fabrication embodiments at every patch orifice.

For fluidic connections to outside tubing, 0.5 mm holes can be punched mechanically into the cured and detached PDMS device. The device can be subsequently bonded to a thin PDMS layer which is spin cast and cured onto a glass substrate. Plastic or other tubes can be connected to the reservoirs, via punched holes, to load both cells and electrolyte solutions and to apply suction to the patch channel.

### Example Alternative Fabrication Methods

The following further example fabrication methods are provided for completeness of this disclosure. It will be recognized from these teachings that many alterations can be made to this method to accommodate different materials and/or methods of manufacturing and/or to provide different configurations as otherwise described herein.

A specific example device according to embodiments of the invention can be fabricated as follows. This fabrication method is similar to that described above. First, a mold is prepared using surface micromachining techniques. As an example, first the narrow patch capillaries are made with 3.1 micron high patterns using deep reactive ion etching. Recording capillaries are 20 microns apart, which allows trapping of, for example, 12 cells along a main channel in a volume of 0.36 nL (150×60×40 micron³ for length, width, and height, respectively). Therefore, in the active device area, the reagent dead volume is 30 pL per recording site. Second, 50 micron high patterns are added for wide connection regions, for example using negative photoresist. After the PDMS (e.g., Dow-Corning Sylgard 184) base and curing agent were mixed (at an example ration of 1:10), the liquid mixture is poured onto the mold and cured at room temperature for 24 hours. For fluidic connections to outside tubing, 0.5 mm holes were punched mechanically into the cured and detached PDMS device. A thin PDMS layer was spin cast on a glass substrate at 3000 rpm for 30 seconds and partially cured (e.g., 90° C. for 1 min.). The device is bonded to the substrate by gently placing the two in contact and fully curing the bottom layer (120° C. for 5 minutes). For use, plastic tubes are connected to the reservoirs, via punched holes, to load both cells and electrolyte solutions and to apply suction to the channel.

Partial cure bonding improves the geometry of the recording capillary by altering the final geometry of the bonded and cured capillary allowing for a tight seal between the cell membrane and the capillary walls even with the hydrophobicity of the PDMS. Partial curing is believed to affect the cross-section of the trapping channel geometry, where instead of providing a square cross-section provides a rounded cross section allowing for a more stable seal.

### 6. Experimental Results

Experimental work was carried out on various of the example devices according to specific embodiments of the invention described throughout this application. In some experiments, a human tumor cell line (HeLa), 12 to 17 μm in diameter, was used for patch clamp seal resistance experiments. Before introducing the cells, the fluidic network was filled with phosphate buffered saline (PBS), taking care to expel all air bubbles. The electrical connection between the reference Ag/AgCl electrode in the main channels and the patch electrode in the lateral patch channel was confirmed by applying a 20 mV square pulse and recording the current response.

FIGS. 11A&B illustrate current response to a 20 mV voltage pulse before (a) and after (b) cell trapping of an example device according to specific embodiments of the invention. Disassociated cells were suspended in PBS and injected into the main channel. Gentle pressure (1 psi) was applied to the patch channel while cells were loaded into the main fluidic channel in order to prevent contamination at the patch site. A cell can either be trapped randomly or selectively by controlling the flow through the main fluidic channel. In specific example experiments with an early design, it was found that a cell within 100-200 μm of the patch channel opening can be trapped within a 1 s time interval by applying 2 psi of negative pressure to the patch channel.

Right after trapping the cell, negative pressure was removed and the cell was allowed to form a seal with the rim of the patch channel. In specific embodiments, membrane protrusion into the patch channel can be seen using standard microscopy and visualization of trapping can be used to control pressure application. In alternative embodiments, cell trapping can be confirmed for example by measuring the resistance at a pore and using the change in measured resistance to confirm the presence of cell and to reduce pressure at that pore where desired.

Patch resistance was recorded by applying a square voltage pulse of amplitude 20 mV and 50 ms duration. The current response was recorded using a standard patch-clamp amplifier and low-pass filtered at 1 kHz. The current response presented contains no capacitance compensation. The resistance of the open patch channel was measured to be 14±4 MΩ. The channel geometry (4 μm·3.1 μm·200 μm) and the conductivity of the electrolyte used (σ=1 S/m) yield a calculated resistance of 17 MΩ, in reasonable agreement with the measurement. Capacitive coupling leads to a current spike when the bias voltage is first applied. Integrating spike currents gives an approximation to the charge stored in the capacitor by: q=∫I dt. Capacitance can then be calculated by using C=q/V. This capacitance measurement method yielded a capacitance of 10±1 pF for connections between the device and the patch clamp amplifier input, but showed no further capacitance increase when the device itself was attached, allowing a conclusion that the device capacitance is within the measurement error, or C_{dev}<1 pF. Calculations, using the device geometry and ∈_{PDMS}=2.46⁹, yielded a predicted device capacitance C_{dev}=0.5 fF.

The current response from the cell by a 20 mV/50 ms current pulse is shown in FIG. 11B. By applying positive pressure to the patch clamp channel, the trapped cell was expelled from the channel. As soon as the cell was expelled, the current response returned to that of the open channel. Subsequent cell trapping in the same channel resulted in lower seal resistance, presumably due to contamination at the opening of the patch channel. In specific embodiments, the invention proposes a single use cell-trapping device, so this contamination does not affect overall usability. In alternative embodiments, a variety of known cleaning techniques could be used to remove contamination from the cell pores.

In experimental results such as with the device illustrated in FIG. 6, electrical connection to the recording capillaries is achieved by inserting Ag/AgCl electrodes into tubing connections outside the active area of the device. The electrodes are in turn connected to the inputs of a multiplexer circuit, which feeds into a headstage of a traditional patch clamp amplifier (in an example system, a customized multiplexor was used in combination with PC-ONE patch clamp components from Dagan, Minn.). The amplifier was driven by custom software (for example, written in a LabView programming environment) and interfaced via an analog to digital conversion board. Prior to each set of experiments, the device is mounted on the stage of an inverted microscope (e.g., Nikon TS100). The microfluidic chambers are filled with electrolyte solution and the electrical connection between the reference and patch electrodes was confirmed by applying a 5 mV square pulse and recording the current response. A typical recording capillary access resistance is in the range of 10-14 MegaOhms. This resistance is higher than the desired 2-5 MegaOhms access resistance of traditional tapered micropipettes. However, because the device channel is not tapered like a heat pulled micropipette, the channel length contributes to the measured access resistance without affecting functionality. For instance, halving the length of the channel generally would halve the measured access resistance without affecting the final seal resistance.

Once the device is filled, adherent cells are trypsinized, spun down at 1000 rpm for 5 minutes, and resuspended in sterile electrolyte solution at a concentration 5×10⁶ cells/ml. A 3 ml syringe is used to inject cells into the main channel. Gentle positive pressure (7 kPa) is applied to the patch channel while cells were loaded into the main fluidic channel in order to prevent contamination at the patch site. A cell can either be trapped randomly or selectively by controlling the flow through the main fluidic channel. A cell found within 100-200 microns of the patch channel opening could be trapped within a 3 s time interval by applying 14-21 kPa of negative pressure to the patch channel. Immediately after trapping the cell, the negative pressure is removed and the cell is allowed to form an electrical seal with the patch channel orifice. Patch array measurements can be performed without the use of vibration isolation equipment.

For both the patch array and traditional electrophysiology, currents were sampled at 5 kHz and filtered with a 2 kHz low-pass Bessel filter. The holding potential was −80 mV and depolarizing stimuli were applied at an interval of 10 s, unless otherwise specified. All signals were post-processed with automated leak subtraction by custom written routines that subtracted a calculated leak current (square wave) from all data traces. The leak resistance was assumed to be constant, and obtained by dividing the resting potential (−80 mV) by the current passed at that voltage.

Both internal and external electrolyte solution contained (mM): 140 KCl, 2 CaCl₂, 2 MgCl₂, 20 HEPES, 10 Glucose. pH was adjusted to 7.3 with KOH and osmolality adjusted to 300 mOsms with glucose.

### Cell Manipulation

In this example configuration, cell trapping was confirmed by light microscopy. The cells are placed in suspension in the central chamber and are sequentially brought to the patch pores by applying negative pressure (28 kPa) to the patch capillaries. In an example system, the total time required for trapping is under three seconds per cell. Trapped cells (shown schematically in FIG. 6A) can be visualized using dark field microscopy as seen in FIG. 6C.

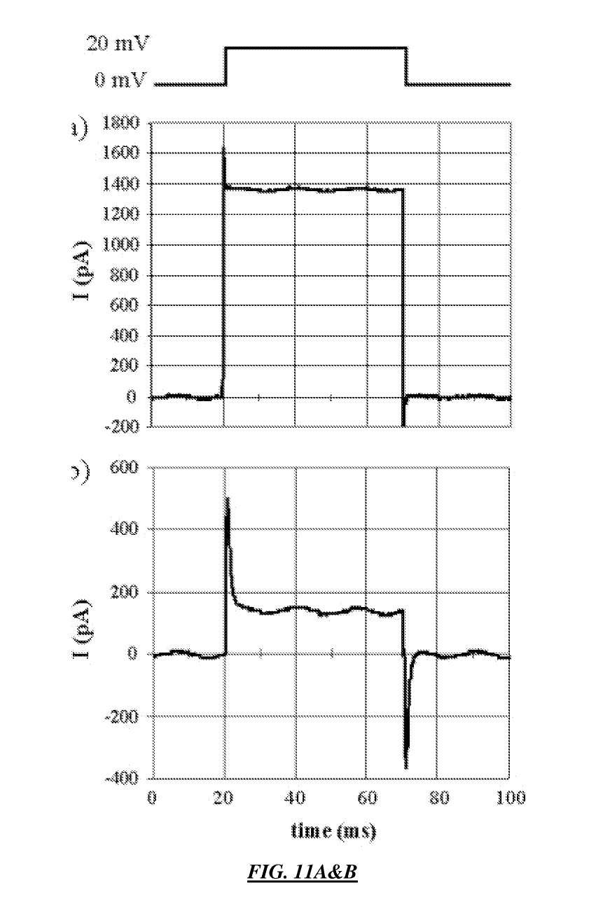

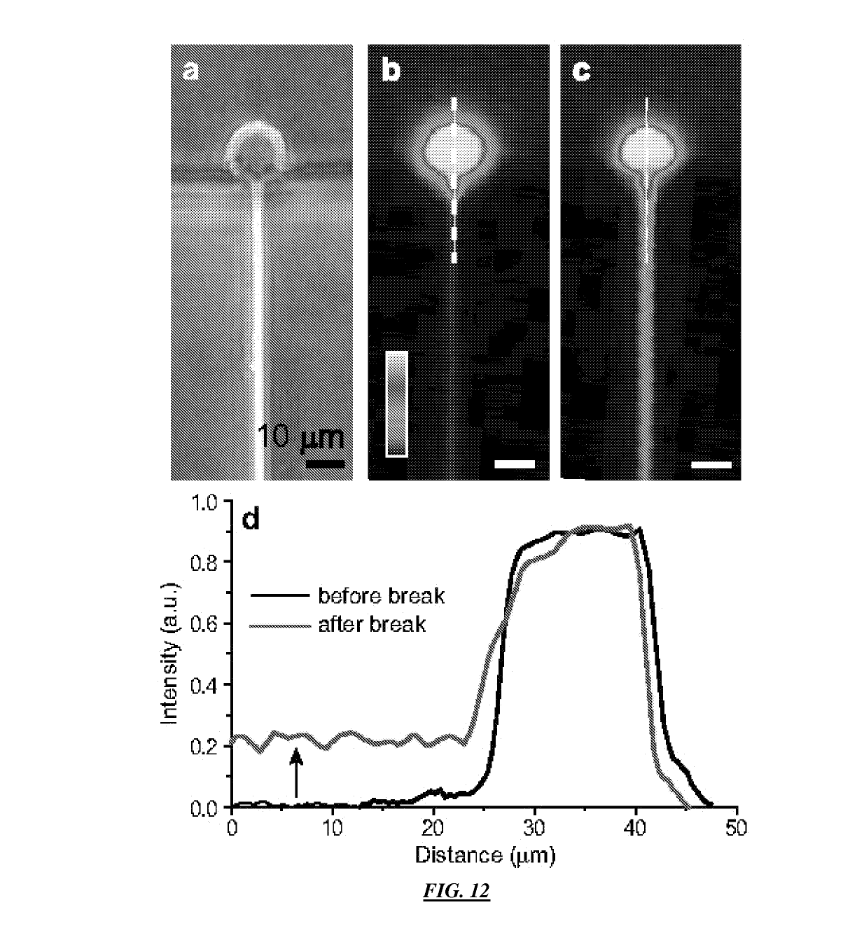

Cells labeled with the cytoplasmic dye calcein were used to quantify both membrane deformation and membrane integrity inside the recording capillary. FIG. 12 illustrates various images of aspects of a cell trapped at the orifice of a capillary according to specific embodiments of the invention. FIG. 12A shows phase contrast image of a cell trapped at the orifice of a recording capillary. FIG. 12B shows fluorescence image of the same cell labeled with the cytoplasmic dye Calcein, showing cell deformation inside the capillary. Dye intensity is represented by a pseudocolor scale. FIG. 12C illustrates that after the application of a negative pressure pulse, the cell deforms further and a break of the membrane patch leads to a whole cell configuration and dye leak into the capillary. FIG. 12D illustrates example graphs of fluorescence intensity as a function of distance (along dotted lines in b, c) show increased deformation and dye diffusion inside the recording capillary (arrow).

As can be seen in the example figures, fluorescent images of trapped cells indicated that the cell membrane routinely protrudes large distances (x>10 ìm) into the channel for a relatively low trapping pressure of 28 kPa. Therefore, seal formation is not restricted to the recording capillary orifice, and can occur several micrometers along the length of the capillary. A pressure spike leads to a membrane break, corresponding to a rise in cellular capacitance. Cytoplasmic access can also be confirmed by observing the diffusion of dye from the cell interior into the recording capillary. The ability to make these types of measurements is a unique advantage of the lateral trapping design, because both the recording capillary and the cell are in the same optical plane. Mechanical and electrical breakdown of the membrane and dye diffusion out of the cell can be quantified, and such data can be used to characterize single cell electroporation on a similar platform.

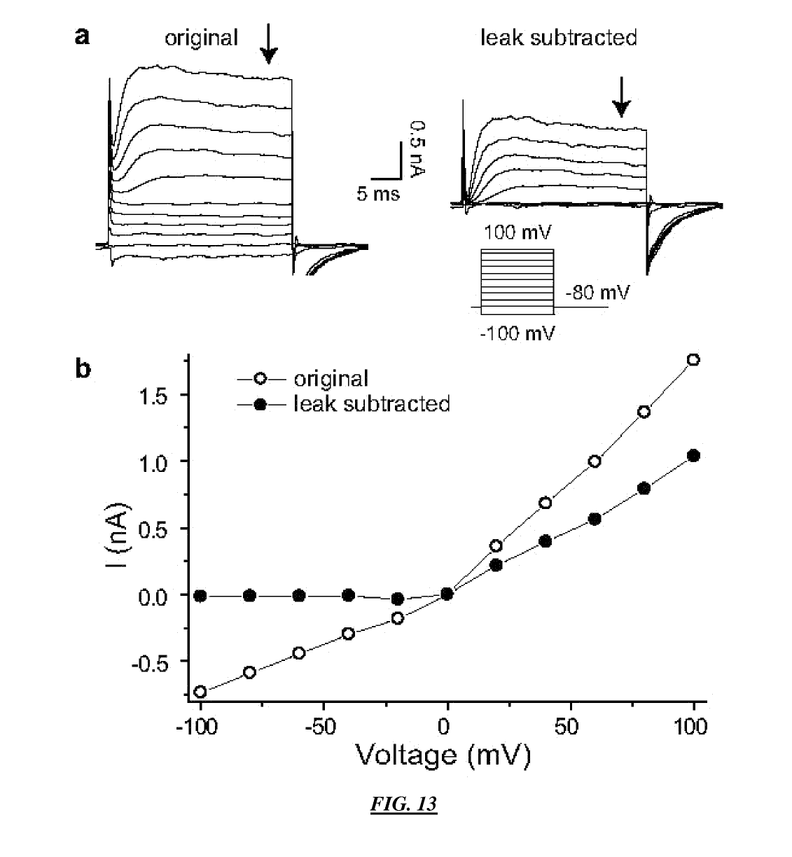

FIG. 13 is a diagram showing an example of whole mammalian cell currents recorded according to specific embodiments of the invention. In this example, whole cell Kv2.1 currents were recorded using a patch clamp array according to specific embodiments of the invention. In FIG. 13A, raw data is shown on the left, and leak subtracted data on the right. The cell membrane potential was depolarized from −100 mV to +100 mV in 20 mV increments for 25 ms. The holding potential was −80 mV. The automated leak subtraction protocol is described in the text. In FIG. 13B, steady-state activation current-voltage relationship for raw (open circles) and leak subtracted (filled circles) data. Data obtained from the end of the depolarizing pulse (arrows, A).

In an array according to specific embodiments of the invention, the recording capillary and the cell substrate are mechanically bonded, eliminating the need for external positioning devices and minimizing the effects of ambient vibration. Early testing has confirmed that seals last for more than about 20-40 min even without the use of vibration isolation equipment, though it is expected that longer seal times can be achieved.

### Giga-Ohm Seals

One parameter of interest in a patch clamp device is its ability to generate gigaohm seals. In early experiments, 27% of trapped cells had an attached resistance of greater than 250 M Ohms (average 301 M), and gigaohm seals were obtained for 5% of cells. Whereas hydrophilic glass-like surfaces were previously believed to be a requirement for giga-seal formation (6, 19), these results demonstrate that the present invention is able to achieve giga-ohm seal formation on hydrophobic elastomer surfaces. Experimental results have also shown that even for more modest sealing resistances (e.g., above about 100 M), it is possible to accurately record whole cell currents down to 20 pA using a device according to the invention.

Given that the cell trapping time is fast, and multiple cells can be trapped simultaneously, the throughput is still acceptable even if selecting only for gigaohm seals. However, further optimization of partial cure bonding protocols and device geometry and fabrication are expected to lead to increases in seal resistance. One technique is to raise the recording channel of the floor of the main chamber to considerably improved seal resistances.

Thus, in specific embodiments, the invention can be embodied as a disposable patch clamp microarray with integrated microfluidics as an electrophysiological measurement tool. Key features of specific example designs include high patch site density, built-in integration with microfluidics, and the ability to study cells by standard microscopy techniques during electrical recording. A micromolded array according to specific embodiments of the invention is capable of mammalian cell recording in a high density format. The device contains an array of lateral capillaries which trap cells efficiently to form tight electrical seals. This scheme has the advantage of integrated microfluidics for compound exchange on both the intracellular and extracellular sides of the cell membrane. The distance between patch sites is 20 μm, a two orders of magnitude reduction in the recording capillary spacing compared to a number of other proposals.

Another convenient feature of this design is the low volume associated with the recording chamber. In one example design, the volume of the main chamber containing 12 cell holding sites is 0.36 nL. By comparison, other planar patch technology can require reagent volumes of 10-100 microL per patch site. Therefore, the reduction in dead volume over such proposals is of order 10⁴. This allows rapid solution exchange to expose attached cells to different reagents in fast succession, with very small reagent consumption and highly uniform solution content between the arrayed patch sites.

In experimental results, the device was successfully used for whole cell recording of the voltage gated channel Kv2.1 expressed by CHO cells. These recordings compare well with traditional patch clamp recordings of the same cell line.

The demonstrated ability for performing fluorescent measurements on multiple patched cells in close proximity can be used in correlating fluorescent data from binding assays, protein expression profiles and intracellular calcium concentration with patch clamp recordings. While similar studies have been performed using traditional pipette patch setups, other high throughput designs are not suitable for this type of combined measurements because the planar substrate is either opaque or requires the use of long working distance objectives.

### 7. Microfluidic Device Design for Fast Application and Removal of Reagents in a Defined Sample Area

In further embodiments, devices and/or methods for the fast application and removal of reagents from a sample area employing microfluidic delivery are provided using some of the novel aspects as described above. It will be understood to those of skill in the art that these aspects can be effectively combined with the cell trapping and/or path clamp techniques described above or separately. Advantages include very simple fabrication by micromolding and the fast (t=30 ms) application and removal of reagent from a given area of the device. The sample area may contain trapped cells, adherent cells on the device substrate, or other reaction loci such as microarray spots. Changes in reagent concentration under controlled time scales is important for high-throughput drug screening applications and well as for understanding fast reaction kinetics.

Pharmacological screening applications typically require the delivery of a given reagent to a test target. The target may be cultured cells (for toxicology, cell viability studies), patch cells (high throughput patch clamp applications), or prepatterned molecules of interest, such as DNA strands (on DNA microarrays) or proteins (proteomics arrays).

Thus, the invention in specific embodiments, provides a simple device and/or method employing microfluidics for the fast application to the target and removal of reagents from the target with superior time scale control and very low dead volumes.

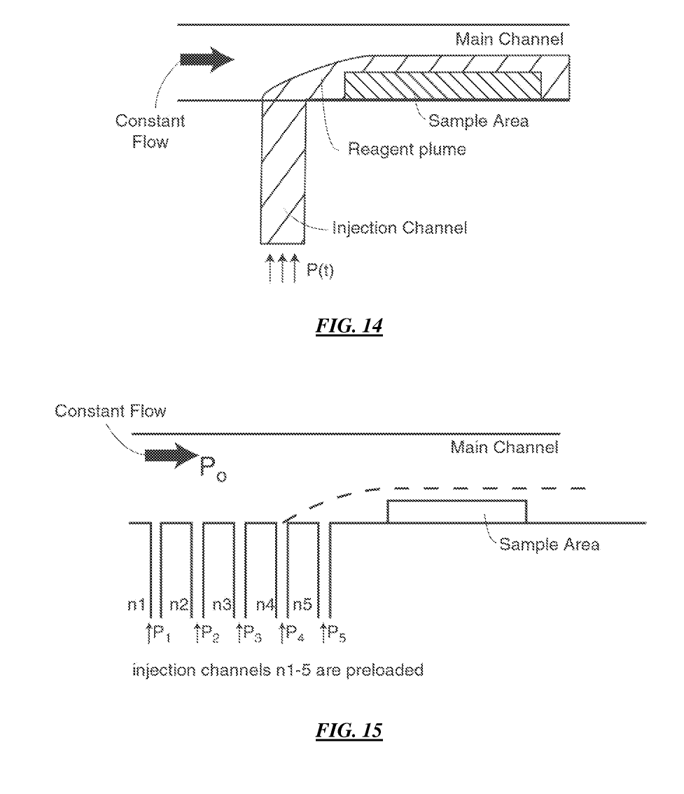

A basic example operation unit of this approach comprises a main channel (containing the reaction target) and an injection channel (used for reagent delivery). A schematic is shown in FIG. 14. In operation, a generally constant flow is supplied to the main channel (e.g., via a syringe pump) and the injection channel is being driven by a pressure (or flow) source at the channel inlet P(t)—pressure as a function of time. When P(t)>P₀ (P₀=the pressure in the main channel), a plume of solution form in the main channel, engulfing the sample area. If P(t)<P₀, no reagent enters the main channel, and the existing plume is removed quickly by the existing flow velocity in the main channel. While the configuration of channels can be varied according to specific embodiments of the invention, one desirable configuration is a lateral configuration where all the channels are in roughly horizontal planes. Thus, this and subsequent related figures can preferable be viewed as top-down view or bottom-up view of a device, with lateral channels therein.

### Applying Multiple Reagents

In specific embodiments, because flow in the main channel follows laminar profiles, the distance between the injection channel and the target sample area is not a critical parameter. Therefore, multiple reagents can be applied by simply arraying a number of injection channels. In FIG. 15, injection channels n1-5 are preloaded with relevant reagents and controlled individually by input pressures P₁₋₅(t). The pressure application can be timed so that only one channel is on at a time, or multiple channels are on simultaneously. In FIG. 15, a situation in which channel n4 is on is displayed. This can be achieved for example by setting P₁₋₃<P₀, P₅<P₀, and P₄>P₀ so that a plume is only present at the outlet of channel 4.

### Applying a Range of Reagent Concentrations

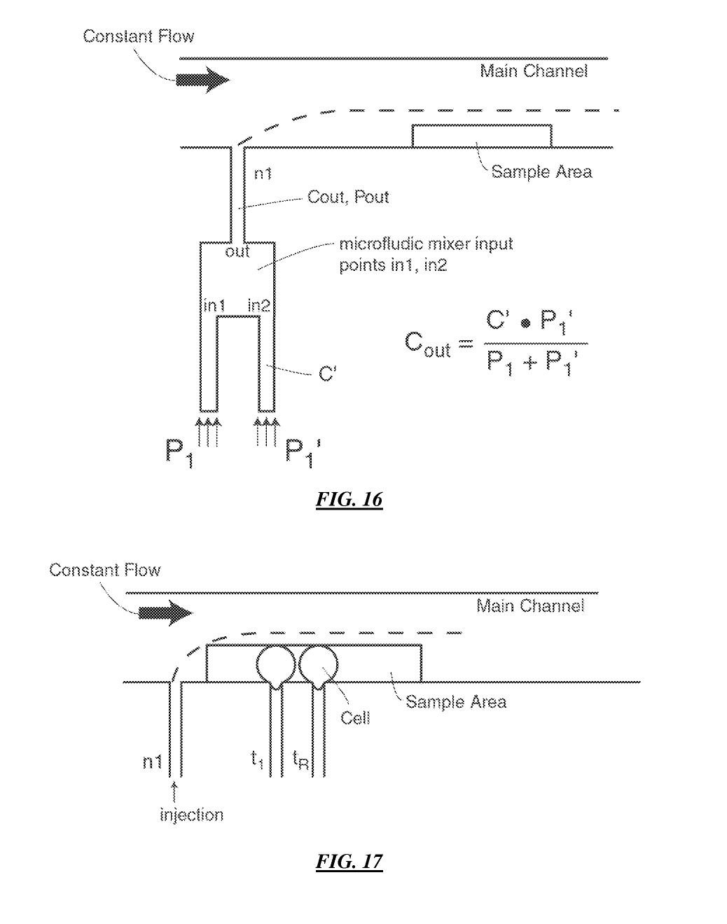

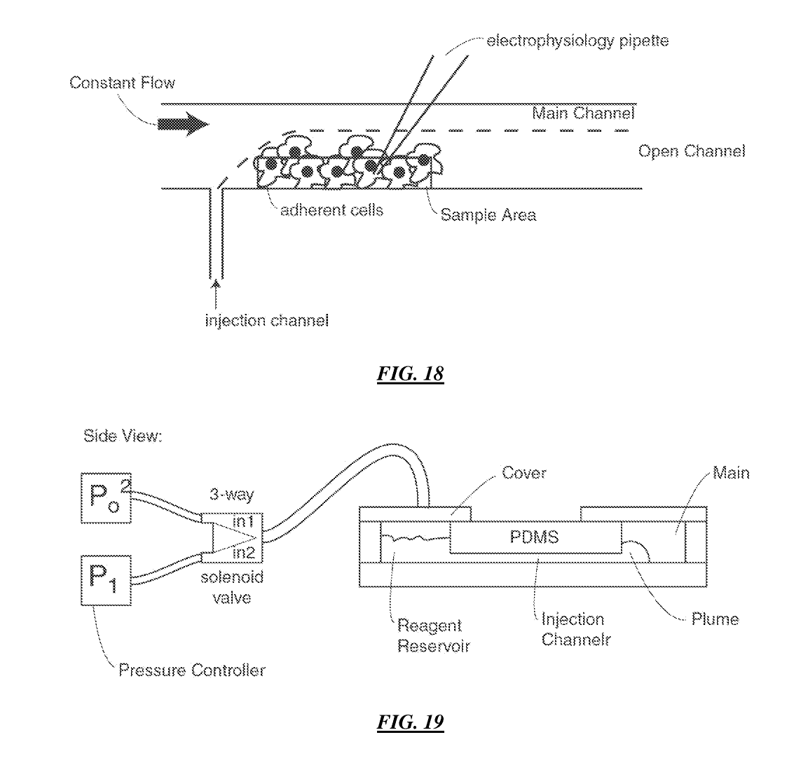

In order to obtain dose response relationships for the interaction of the reagents applied with the target sample, it is useful to be able to apply an arbitrary concentration of reagent to the target sample area. Such a setup is shown in FIG. 16. Upstream of the injection channel, a microfluidic mixer can be used in order to obtain a desired reagent concentration at the outlet. One inlet is connected to the reagent reservoir, while the other is connected to a reservoir of stock solution. In this figure, the concentration Cₒᵤₜ at the output n1 is a simply calculation of the pressures and concentrations of the two inputs P₁ and P₁′.

### Reagent Delivery to Cellular Samples

A variety of cell-based assays (electrophysiology, viability, membrane integrity) require the delivery of reagents to cells (ie sample target=mamallian cells). In order to apply and remove reagents quickly from cellular samples, cells must be immobilized in the sample target area. According to specific embodiments of the invention, this may be achieved by cell trapping at the intersection of trapping channels with the main fluidic channels as described herein (FIG. 17). These trapping sites can be arrayed, and after an experiment trapped cells can be expelled by applying positive pressure to the trapping channels, so that fresh cells may be trapped for subsequent experiments. While cells are trapped in the sample area, reagents may be applied as described above.

### Adherent Cells

According to further specific embodiments of the invention, cells can also be immobilized in the sample area by culturing cells in the main channel of the device. A cell suspension may be introduced into the main channel, and cells allowed to settle and attach to the substrate. After attachment, media can be flowed in the main channel in order to keep the cells fed and oxygenated. After cells are firmly attached to the substrate in the sample area, desired reagents may be applied to those cells as described above. In specific examples, by constructing an open top channel, glass pipettes can be introduced for traditional electrophysiological recording from the adherent cells in the target area. Alternatively, electrophysiological recording can be performed using integrated systems or devices as described above.

### Variable Pressure Application to the Injection Channel.

According to further specific embodiments of the invention, fast switching of injection channel from ON to the OFF configurations requires fast switching of the pressure applied to the injection channel inlet from P_{ON}>P₀ to P_{OFF}<P₀, where P₀ is again the pressure in the main channel. This can be achieved through the use of a fast 3-way solenoid valve (for example a Lee valve) as shown in FIG. 19. One input of the valve is connected to atmospheric pressure (ie P_{OFF}), while the other is connected to a pressurized reservoir (P_{ON}). The output of the valve is connected to the reagent reservoir feeding the injection channel as shown.

### 8. Array of Hollow Cylindrical Electrodes for Microfluidic and Electric Interface

According to further specific embodiments of the invention, an arrangement of hollow Ag/AgCl cylindrical electrodes can be used with microfluidic systems such as the patch clamp system described above to serve as both a fluidic interface and an electrical interface for microfluidic chips. In specific embodiments, as fluid flows through these hollow electrodes, electrical and fluidic connections are established. This eliminates the need for fragile, cumbersome, and expensive Ag/AgCl pellet electrodes that have often been used in patch clamp applications and also minimizes microfluidic circuitry.

Reusability and ease of manufacturing are key advantages an electrode array according to specific embodiments of the invention over imbedded on-chip electrodes. According to specific embodiments of the invention, the interface allows the inexpensive polymeric microfluidic devices to be disposable, while the more expensive mating electrodes can be easily detached and reused.

Polymeric patch clamp devices, such as those described herein, can be grouped into arrays or other configurations. In many situations, pellet electrodes are cumbersome to use with the devices and are expensive (costing ˜$10 each). While the devices themselves can be manufactured for just pennies a piece, the high cost of the electrodes is the cost-limiting factor. Additionally, the delicate pellet electrodes tend to break easily, especially because they are jammed into the fluidic tubing while the fluid must flow around them.

While electrodes made of noble metals can be easily deposited onto a glass substrate, Ag/AgCl is more difficult. Integrated thin film Ag/AgCl electrodes have been demonstrated but have their drawbacks. (For example, see Kojima, K and Taura, T, Thin-Film Ag/AgCl Structure and Operational Modes to Realize Long-Term Storage, Journal of The Electrochemical Society, 2001, 148 (12), pp. E468-E474). DuPont has developed a Ag/AgCl paste (BQ166) that can be screen-printed, but it suffers from poor-adhesion to glass substrates. Even if integrating on-chip Ag/AgCl electrodes could be done more efficiently, it would never-the-less significantly increase the manufacturing costs of the devices. When using disposable polymeric devices, the costly integrated electrodes would be unnecessarily disposed of after a single use.

According to specific embodiments of the invention, low cost electrodes are provided that can efficiently mate with various microfluidic systems, including patch clamp systems described herein. Such electrodes are critical, for example, to physiological measurements for high through-put screening. The invention, therefore, according to specific embodiments, routes fluid flow through instead of around the electrodes using a detachable Ag/AgCl array, for example built on a printed circuit board (PCB). These electrodes, according to specific embodiments of the invention, serve not only as electrical connections, but fluidic conduits as well.

In specific examples, fluid flows through the hollow Ag/AgCl electrodes that connect the device to tubing that then connects to a syringe for sample loading. The configuration of the electrodes, as well as additional processing capabilities, can be specifically designed to mate with the microfluidic device.

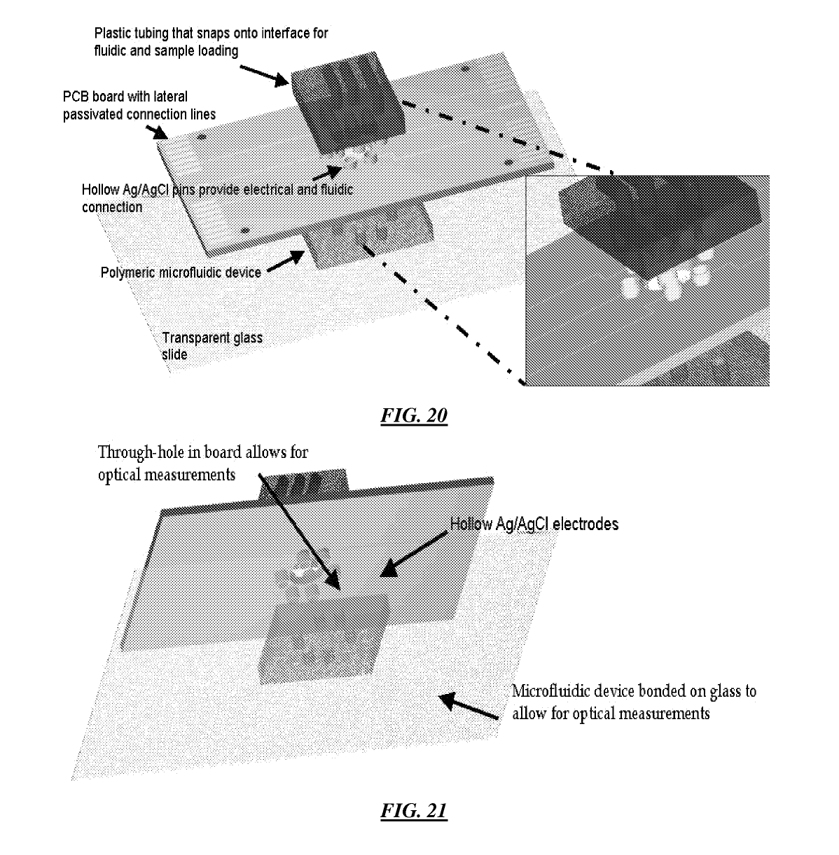

FIG. 20 illustrates an example schematic of a hollow electrode interface (in this example configured on a PCB) mating with device and tubing according to specific embodiments of the invention. In this figure, the conduit/electrodes are embedded in a detachable PCB interface that includes passivated lateral connectors for easy electrical connection to other equipment, as will be understood in the art. In these example figures, a circular arrangement of six electrode/conduits are shown, which are arranged to mate with channel connections on a microfluidic devices and optionally also with an external source of fluidics. This is only an example arrangement, however, and any other convenient arrangement is possible.

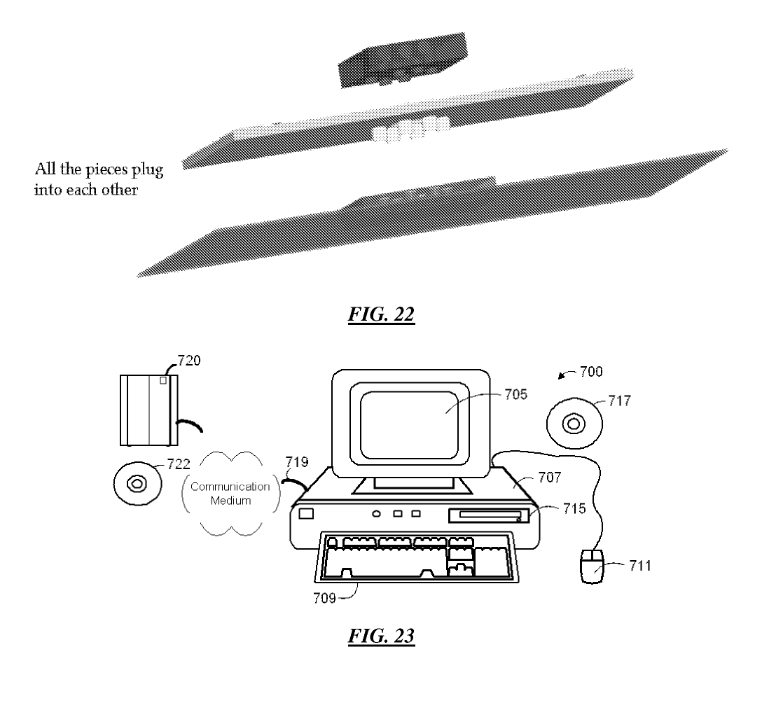

FIG. 21 illustrates a bottom view of an example hollow electrode interface according to specific embodiments of the invention. FIG. 22 illustrates an exploded side view of tubing, interface and device assembly of an example hollow electrode interface according to specific embodiments of the invention. In this view, it can be seen that the three elements connect, allowing for an easy to use microfluidics/electrophysiological measuring system with an exchangeable/disposeable elements.

### 9. Diagnostic and Drug Development Uses

As described above, following identification and validation of a assay for a particular cellular process, in specific embodiments devices and/or systems as described herein are used in clinical or research settings, such as to screen possible active compounds, predicatively categorize subjects into disease-relevant classes, text toxicity of substances, etc. Devices according to the methods the invention can be utilized for a variety of purposes by researchers, physicians, healthcare workers, hospitals, laboratories, patients, companies and other institutions. For example, the devices can be applied to: diagnose disease; assess severity of disease; predict future occurrence of disease; predict future complications of disease; determine disease prognosis; evaluate the patient's risk; assess response to current drug therapy; assess response to current non-pharmacologic therapy; determine the most appropriate medication or treatment for the patient; and determine most appropriate additional diagnostic testing for the patient, among other clinically and epidemiologically relevant applications. Essentially any disease, condition, or status for which a cellular characteristic measurable using a patch clamp has been identified can be evaluated.

### Web Site Embodiment

The methods of this invention can be implemented in a localized or distributed data environment. For example, in one embodiment featuring a localized computing environment, a patch clamp device according to specific embodiments of the present invention is configured linked to a computational device equipped with user input and output features. In a distributed environment, the methods can be implemented on a single computer, a computer with multiple processes or, alternatively, on multiple computers.

### Kits

A device according to specific embodiments of the present invention is optionally provided to a user as a kit. Typically, a kit of the invention contains one or more patch clamp devices constructed according to the methods described herein. Most often, the kit contains a diagnostic sensor packaged in a suitable container. The kit typically further comprises, one or more additional reagents, e.g., substrates, tubes and/or other accessories, reagents for collecting blood samples, buffers, e.g., erythrocyte lysis buffer, leukocyte lysis buffer, hybridization chambers, cover slips, etc., as well as a software package, e.g., including the statistical methods of the invention, e.g., as described above, and a password and/or account number for accessing the compiled database. The kit optionally further comprises an instruction set or user manual detailing preferred methods of using the kit components for sensing a substance of interest.

When used according to the instructions, the kit enables the user to identify disease specific cellular processes. The kit can also allow the user to access a central database server that receives and provides expression information to the user. Such information facilitates the discovery of additional diagnostic characteristics by the user. Additionally, or alternatively, the kit allows the user, e.g., a health care practitioner, clinical laboratory, or researcher, to determine the probability that an individual belongs to a clinically relevant class of subjects (diagnostic or otherwise). In HTS, a kit according to specific embodiments of the invention can allow a drug developer or clinician to determine cellular responses to one or more treatments or reagents, either for diagnostic or therapeutic purposes.

### Embodiment in a Programmed Information Appliance

The invention may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

### Integrated Systems

Integrated systems for the collection and analysis of cellular and other data as well as for the compilation, storage and access of the databases of the invention, typically include a digital computer with software including an instruction set for sequence searching and/or analysis, and, optionally, one or more of high-throughput sample control software, image analysis software, collected data interpretation software, a robotic control armature for transferring solutions from a source to a destination (such as a detection device) operably linked to the digital computer, an input device (e.g., a computer keyboard) for entering subject data to the digital computer, or to control analysis operations or high throughput sample transfer by the robotic control armature. Optionally, the integrated system further comprises an electronic signal generator and detection scanner for probing a patch clamp device. The scanner can interface with analysis software to provide a measurement of the presence or intensity of the hybridized and/or bound suspected ligand such as by measurement of electrical characteristics of the cell membrane.

Readily available computational hardware resources using standard operating systems can be employed and modified according to the teachings provided herein, e.g., a PC (Intel x86 or Pentium chip-compatible DOS,™ OS2,™ WINDOWS,™ WINDOWS NT,™ WINDOWS95,™ WINDOWS98,™ LINUX, or even Macintosh, Sun or PCs will suffice) for use in the integrated systems of the invention. Current art in software technology is adequate to allow implementation of the methods taught herein on a computer system. Thus, in specific embodiments, the present invention can comprise a set of logic instructions (either software, or hardware encoded instructions) for performing one or more of the methods as taught herein. For example, software for providing the data and/or statistical analysis can be constructed by one of skill using a standard programming language such as Visual Basic, Fortran, Basic, Java, or the like. Such software can also be constructed utilizing a variety of statistical programming languages, toolkits, or libraries.

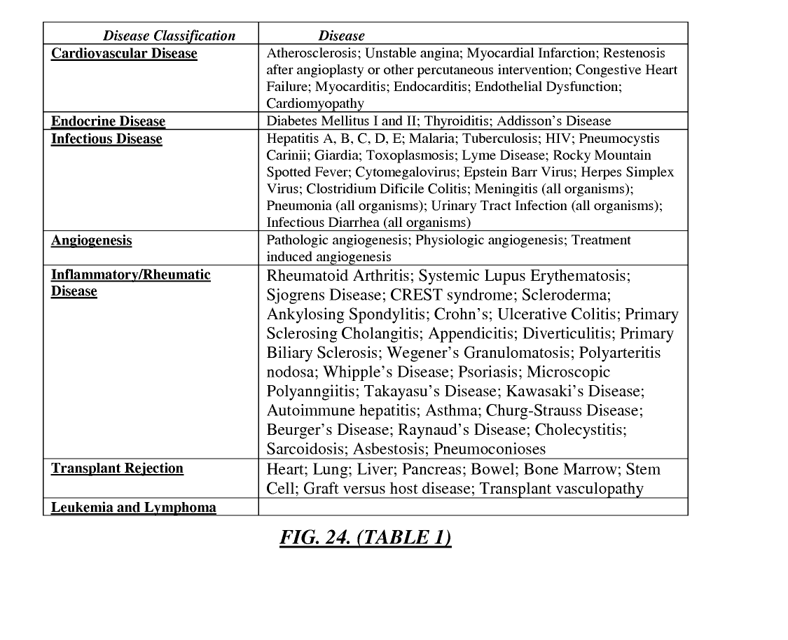

FIG. 23 is a block diagram showing a representative example logic device in which various aspects of the present invention may be embodied. FIG. 23 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection.

Various programming methods and algorithms, including genetic algorithms and neural networks, can be used to perform aspects of the data collection, correlation, and storage functions, as well as other desirable functions, as described herein. In addition, digital or analog systems such as digital or analog computer systems can control a variety of other functions such as the display and/or control of input and output files. Software for performing the electrical analysis methods of the invention are also included in the computer systems of the invention.

Optionally, the integrated systems of the invention include an automated workstation. For example, such a workstation can prepare and analyze samples by performing a sequence of events including: preparing samples from a tissue or blood sample; exposing the samples to at least one patch clamp device comprising all or part of a library of candidate probe molecules; and detecting cell reactions by various electrical measurements. The cell reaction data is digitized and recorded in the appropriate database.

Automated and/or semi-automated methods for solid and liquid phase high-throughput sample preparation and evaluation are available, and supported by commercially available devices. For example, robotic devices for preparation of cells. Alternatively, or in addition, robotic systems for liquid handling are available from a variety of sources, e.g., automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Beckman Coulter, Inc. (Fullerton, Calif.)) which mimic the manual operations performed by a scientist. Any of the above devices are suitable for use with the present invention, e.g., for high-throughput analysis of library components or subject samples. The nature and implementation of modifications to these devices (if any) so that they can operate as discussed herein will be apparent to persons skilled in the relevant art.

### Other Embodiments

Although the present invention has been described in terms of various specific embodiments, it is not intended that the invention be limited to these embodiments. Modification within the spirit of the invention will be apparent to those skilled in the art.

It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this submission, including any references filed as part of an Information Disclosure Statement, are incorporated by reference in their entirety.

1. (canceled)

2. A method of fabricating an integrated patch clamp device comprising:
preparing a mold by making height patterns defining narrow patch channels using deep etching;
adding patterns for wide connection regions;
introducing a settable material into the mold and curing;
detaching the set material from the mole;
placing holes for connection of tubes;
connecting tubes to reservoirs, via said holes, to load cells and/or electrolyte solutions and to apply suction to patch channel.

3. The method of claim 2 further wherein:
said mold is constructed from one or a combination of:
silicon;
ceramic;
metal or metal alloy.

4.-6. (canceled)

7. The method of claim 2 further wherein:
said patterns defining the narrow patch channels are formed using deep reactive ion etching; and further patterns are added for wide connection regions using photoresist.

8. The method of claim 2 further wherein:
said moldable material comprises polydimethylsiloxane (PDMS) and a curing agent.

9. The method of claim 2 further comprising:
subsequently bonding a molded device to a thin PDMS layer which was spin cast and then cured or partially cured onto a glass substrate.

10. (canceled)

11. A cell trapping device comprising:
a substrate;
a main reservoir able to hold cells in a fluidic medium;
at least one lateral opening in a side of said main reservoir;
at least one trapping channel operatively connected to said at least one lateral opening;
such that a cell in said main reservoir can be selectively immobilized at said lateral opening by negative pressure in said trapping channel.

12. The device according to claim 11 further wherein:
said substrate is a three dimensional structure comprising a length, a width and a thickness, said thickness being a smallest dimension; and
said side of said main reservoir is roughly parallel to said thickness.

13. (canceled)

14. The device according to claim 11 further comprising:
at least two electrical connections for measuring electrical characteristics between said main reservoir and said trapping channel.

15. The device according to claim 11 further wherein:
said lateral opening has effective dimensions of less than about 3 microns by 3 microns.

16. The device according to claim 11 further comprising:
at least three lateral openings in said main channel, said lateral openings spaced less than 40 microns apart.

17. (canceled)

18. The device according to claim 16 further wherein:
said lateral openings are electrically connected to operate as independent patch channels and are arranged in a horizontal plane allowing multiplexed parallel patch sites that are less than 30 microns apart.

19. The device according to claim 17 further wherein:
patch channels are in a horizontal plane with multiplexed parallel patch sites having a distance between patch sites of between one hundred μm and one thousand μm.

20. (canceled)

21. A multiple cell trapping device according to claim 11 further comprising:
a main reservoir able to hold cells in a fluidic medium running parallel to the largest dimensions of said substrate;
a plurality of lateral openings in sides of said main reservoir, at least some of said openings operatively connected to a plurality of trapping channels;
a microfluidic input for introducing cells in a fluid to said main reservoir;
one or more microfluidic trapping connections for applying negative pressure to said lateral openings;
such that cells in said main reservoir can be selectively immobilized at said lateral openings.

22. The device according to claim 21 further wherein:
said substrate is formed of an elastomer;
said lateral openings have a cross section less than about 3 microns by 3 microns; and
said lateral openings are operatively connected to trapping channels with cross sections less than about 3 microns by 3 microns.

23.-24. (canceled)

25. A device allowing fast application and removal of reagents from a sample area employing microfluidic delivery comprising:
a sample area;
a main channel; and
one or more an injection channels;
wherein in operation, a generally constant fluid flow is supplied to the main channel and said injection channel is being driven by a pressure as a function of time.

26. The device according to claim 25 further wherein said sample area may contain trapped cells, adherent cells on the device substrate, and/or other reaction loci such as microarray spots.

27. (canceled)

28. The device according to claim 25 further wherein:
said main channel and said injection channels have a lateral configuration where all the channels are in roughly horizontal planes;
said one or more injection channels comprise an array of a number of injection channels.

29.-30. (canceled)

31. A device for connecting a microfluidic assay chip to external electrical and fluidic systems comprising:
an arrangement of hollow cylindrical electrical conductors connected to a plurality of electrical connectors.

32. The device according to claim 31 further wherein:
said conductors are arranged so as to operatively mate with fluidic connections on said assay chip;
said conductors are arranged so as to operatively mate with fluidic couplings to an external fluidic system;
said electrical connectors are arranged so as to operatively mate with an electrical socket of an electronic testing system;
as fluid flows through said hollow electrodes, electrical and fluidic connections are established; and
said hollow electrodes are reusable with multiple microfluidic chips.

35. The device according to claim 31 further wherein:
said hollow cylindrical electrical conductors are comprised of one or more of:
Ag/AgCl;
a metal/metal-chloride alloy;
a conducting polymer;
a metal;
a conducting ceramic.

36.-40. (canceled)

41. (canceled)

42. (canceled)

43. (canceled)
